(19) United States
(12) Patent Application Publication
(10) Pub. No.: US 2012/0094308 A1
(43) Pub. Date: Apr. 19, 2012

(54) KIT AND METHOD FOR THE PREMORTEM IN VITRO DETECTION OF ALZHEIMER'S DISEASE

(75) Inventor: Celia Sanchez Ramos, Madrid (ES)

(73) Assignee: UNIVERSIDAD COMPLUTENSE DE MADRID, Madrid (ES)

(21) Appl. No.: 13/377,751

(22) PCT Filed: Jul. 23, 2009
(86) PCT No.: PCT/ES09/00392

§ 371 Date: Dec. 12, 2011

(30) Foreign Application Priority Data
Jul. 23, 2009 (ES) P20090001632

(51) Int. Cl. G01N 21/64; G01N 33/82; G01N 33/566

(52) U.S. Cl. 435/7.5; 436/501; 435/7.92; 435/7.93; 435/7.94; 435/7.95

(57) ABSTRACT

Kit and method for the pre mortem in vitro detection of Alzheimer's disease The invention relates to a detection kit for Alzheimer's disease based on the vitro identification of the presence of biomarkers in the remains normally discarded after crystalline lens surgery. The kit can be used for the pre mortem detection of the disease, even before the appearance of clinical symptoms. Among the available biomarkers, the biomarker of choice is beta-amyloid peptide, for which several methods of sample processing and labelling for the presence of the biomarker may be used. Embodied in the invention is also the use of the crystalline lens remains discarded during surgery, often cataract surgery, to prepare a method for the in vitro pre mortem detection of Alzheimer's disease.

### TECHNICAL FIELD OF THE INVENTION

The invention relates to the early detection of Alzheimer's disease (AD) using the remains of the crystalline lens discarded during surgery. To date, the only reliable diagnostic test for this disease is examination of patient brain tissue following death. This invention has been designed for the early identification of patients with AD, preferably before any signs of brain disease appear.

### BACKGROUND ART

Alzheimer's disease (AD) is a neurodegenerative disorder that manifests with impaired cognitive function and abnormal behaviour patterns, Typically, AD is characterized by a gradual loss of memory and other mental capacities, as more and more neurons the and the different brain areas undergo atrophy.

The impacts of AD are such that estimates of affected persons in the world run at 18 to 22 million, along with a mean prevalence of 3-15% and yearly incidence of 0.3-0.7%. Prevalence varies but it has been calculated that 1-5% of persons older than 65 years and 20-40% of those over 85 years have AD. In effect, its prevalence doubles every 5 years after the age of 65 years and it accounts for 50 to 60% of all dementias detected in postmortem studies (J. Vilalta-Franch, S. López-Pousa, J. Garre-Olmo, A. Turón Estrada e I. Pericot-Nierga Heterogeneidad clinica de la enfermedad de Alzheimer según la edad de inicio Revista de Neurologia 2007; 45:67-72).

Although there is presently no cure for this disease, the drugs available today to treat AD can help preserve a patient's mental skills over months or years. The course of AD, however, will essentially be unchanged. Thus, an early diagnosis of AD will increase the chances of successful treatment.

There is currently no reliable specific diagnostic method available for AD and its diagnosis is in a first instance based on the patient's clinical history and on observation of neurological and psychological signs on the part of both relatives and healthcare personnel. In second place, brain imaging techniques are pursued mainly computerized tomography (CT), nuclear magnetic resonance (NMR), positron emission tomography (PET) or single photon emission CT (SPECT). These techniques are able to detect different signs that indicate some type of dementia but they do not provide a definitive diagnosis (Manuela Neumann, Deepak M. Sampathu, Linda K. Kwong. Ubiquitinated TDP-43 in frontotemporal lobar degeneration and amyotrophic lateral sclerosis. Science 2006; 314:130-133).

The accuracy of a diagnosis of AD based on clinical observations, neurological and neuropsychological tests and brain imaging techniques approaches 85%, but the final diagnosis requires histological procedures on brain tissue specimens obtained during autopsy. This means that a definitive diagnosis of AD can only be made after the patient has died.

Postmortem studies have served to establish two abnormal structures in the AD brain. One is a deposit of a protein fragment β-amyloid (βA) peptide, that forms the so-called senile or neuritic plaques, and the other is the twisted fibres of another protein forming so-called neurofibrillary tangles in neurons.

The senile plaques referred to in AD are composed of a protein core surrounded by degenerated neurons along with glial and microglial cells. βA peptide is normally produced in monomeric soluble form and circulates in low concentrations in the cerebral spinal fluid (CSF) and blood. Despite its seemingly detrimental role in this disease, βA peptide takes part in normal cell functions:
it has an autocrine function in stimulating cell proliferation.
it promotes cell adhesion and protects neurons against oxidative damage.
in physiological concentrations, it could act as a neurotrophic and neuroprotective factor.
it is a physiological regulator of the activity of potassium (K) and calcium (Ca) ion channels in neurons and is secreted by some of these cells in response to neuronal activity in the negative regulation of excitatory synaptic transmission.
its deposits may trap potentially dangerous metal ions (Selkoe D J. Cell biology of the amyloid β-protein precursor and the mechanism of Alzheimer's disease. Annu Rev Cell Biol (1994); 10:373-40)

Neurofibrillary tangles are the remains of damaged microtubules (microtubules form the neuron structure through which nutrients flow). Tangles first form in the hippocampus, which is the brain region that deals with memory. Disruption of the microtubular system leads to defects in axon transport and probably to cell degeneration. The latter has been observed in neurons affected by AD, This process involves tau proteins of the MAP (microtubule-associated proteins) family such that diseases in which neurofibril formation is observed are often referred to as tauopathies (Castaño, Frangione. Biology of disease: Human amyloidosis, Alzheimer's disease and related disorders. Laboratory Investigation (1998) 58:122).

In the cytoplasm, the tau protein is normally phosphorylated. This means that joined to it, it has two phosphate groups that play an important role in regulating the function of the protein. Phosphorylation confers the tau protein the ability to stabilize microtubules and regulate the outgrowth of axons and neurons. Aggregates of tau form helical filaments, which unlike the normal protein, feature a large number of phosphates in a hyperphosphorylated form (Wang H. Y., LiVV., Benedetti N. J. and Lee D. H. Alpha 7 nicotinic acetylcholine receptors mediate beta-amyloid peptide-induced tau protein phosphorylation. J, Biol. Chem. (2003) 278, 31 547-31 553),

In AD and other tauopathies, abnormal phosphorylation and/or hyperphosphorylation occur and neither the soluble or filament forms of hyperphosphorylated tau are capable of inducing microtubule formation or stabilizing existing microtubules. In addition, aberrant forms of tau inhibit tubulin assembly within microtubules and can disassemble those formed with the normal protein.

For several years, researchers have searched for possible biomarkers in biological fluids such as CSF, blood and urine on which to base tests for the diagnosis of AD. To date, however, experts have not been able to establish any marker-based protocol for the premortem detection of AD in these types of sample (H. M. Schipper. The role of biologic markers in the diagnosis of Alzheimer's disease. Alzheimer's & Dementia (2007) 3:325-332). Hence. present 3A peptide and tau protein are the main known biomarkers for the in vitro detection of AD.

Patent EP1420830A1 describes an in vivo ocular AD diagnosis procedure based on the use of compounds, or markers, that bind to βA proteins present in the eye tissues of patients. These markers are generally fluorophores such that the fluorescence emitted upon binding to βA protein can be detected and quantified indicating the presence or not of the disease in a patient. The procedure proposed is the application of these markers to the eye in the form of a liquid or gel. The lipophilic nature of these compounds helps their penetration in the eye tissue where they bind to βA peptide. After a sufficiently long time has elapsed to ensure the marker's penetration and binding, fluorescence is directly measured in the patient's eyes. This method is a premortem, noninvasive AD diagnostic test.

In contrast, patent EP1913866A1 promulgates a new noninvasive way to detect biological markers of AD in the crystalline lens and other eye tissues using, quasi-elastic light dispersion, Raman spectroscopy, fluorimetry or other optical techniques. These techniques allow the detection and monitoring of βA peptide deposition in the eye for the diagnosis of neurodegenerative disorders including AD.

Despite the efforts and developments described above, the unequivocal diagnosis of AD is still only possible using eye tissue specimens obtained postmortem. It is therefore essential that new tools are developed for the early, preferably pre-clinical, detection of AD, to enable its adequate treatment in the early stages of disease and delay the onset and progression of its symptoms.

### DISCLOSURE OF THE INVENTION

One aspect of the present invention relates to an AD detection kit for use on fragments of the crystalline lens discarded during surgical procedures in which the ocular lens is extracted. In these remains, is determined the presence of an AD biomarker, preferentially βA peptide, which besides being deposited as plaques in the brains of patients with AD also does so in the crystalline lens.

Another embodiment of the invention relates to the use of discarded remains obtained during operations on the crystalline lens to prepare a method of AD detection through the determination of a biomarker, for example, βA peptide.

In ophthalmologic medicine, the, full or partial opacification of the crystalline lens is termed cataract. Today, the most frequent surgical intervention perfomed on the crystalline lens is cataract surgery. Cataract is a chronic disease associated with the ageing process whose prevalence has substantially increased along with the increasing life expectancy of the population. Cataracts may develop for several reasons, though acquired cataract is the most common type and the leading cause of vision loss among persons older than 55 years. This means that cataract surgery is a very frequent intervention in the age group of persons susceptible to develop AD. Effectively, in persons under the age of 50-55 years, the prevalence of cataract is low, around 0.2% to 7%; in intermediate age groups, between 55 and 65 years, around 20% are affected; and in persons aged 70-75 years, between 40% to over 60% are affected. Even before any serious vision problems requiring surgery, over 75% of persons older than 60 years and 95% of persons older than 75 years show some degree of crystalline lens opacity.

Among the different surgical procedures available to remove a cataract, the, most widely used, extracapsular surgery, generates fewer complications and allows the implant of an intraocular lens. In this type of surgery, only the opaque portion of the cataract is removed while preserving the posterior portion of the capsule, or lens sac. This structure serves as a support for the intraocular lens, which will occupy the same site as the extracted natural lens. Extracapsular surgery is generally undertaken using a technique known as phacoemulsification in which a small incision is made in the eye tissue, followed by destruction of the opaque crystalline lens of the patient. This destruction is achieved by ultrasound waves that cause a vibration of 30,003 to 60,000 times per second. This vibration, breaks the cataract up into sufficiently small fragments that are then emulsified and gently aspirated. The phacoemulsification device consists of a physiological saline flow system for irrigation and aspiration connected to the ultrasound probe, itself. This system is used to aspirate the emulsified fragments and also cools down the tip of the emulsifier to avoid burns. The fragments of the aspirated opaque crystalline lens are collected with the physiological saline into a vessel and these remnants are then discarded. The operation ends with the placement of an intraocular lens at the site previously occupied by the natural lens of the patient (Olitsky S E, Hug D, Smith L P. Abnormalities of the Lens. In: Kliegman R M, Behrman R E, Jenson H B, Stanton B F, eds. Nelson Textbook of Pediatrics, 18^{va} ed. Philadelphia, Pa: Saunders Elsevier; 2007; cap. 627).

The AD detection kit of this invention has two components. First, it comprises a recipient where the remains of the crystalline lens are collected, after surgery. These remains are usually found in physiological saline. Second the kit comprises a labelling agent that binds to the AD biomarker present in the crystalline lens remains. The kit also includes a system for the detection of the labelling agent bound to the biomarker, to determine the presence or absence of this biomarker and thus of AD in the patient.

Additionally, the kit could include a system to separate the solid from the liquid components of the mixture of lens remains obtained from surgery. This is generally a centrifuge that subjects the mixture to a rotation motion whose force is of greater intensity than that of gravity, causing the solid remains to sediment out of the mixture.

Using the discarded remains of crystalline lens operations, the kit permits the detection of the amount of biomarker present in the crystalline lens of the patient as the consequence of the onset and progression of AD. The present invention also relates to the method of in vitro detection of an AD biomarker in the crystalline lens. This method comprises a collection step in which the crystalline lens remains removed, during cataract surgery along with the physiological saline used in the operation are collected in the recipient. This mixture can be directly treated with labelling agents that bind to the biomarker or can be centrifuged to separate the solid, or fragmented cells, from the liquid, or physiological saline, in the mixture. Once separated, the solid portion is, processed for labelling for the given AD biomarker selected to detect the disease in the patient. This step varies depending on the technique selected to detect the biomarker. Thus, this technique could comprise an embedding step in which the specimen samples are embedded in paraffin blocks to obtain cuts or thin sections to which the labelling agent of the selected AD marker is applied. Blocks may also be obtained by rapid freezing of the solid part of the sample. Another option is to separate the proteins from the rest of the elements of the solid portion using any conventional method (sonication, gentle detergent treatment, etc.) and then determine the presence or absence of the biomarker in the protein mixture using any of the techniques used by experts in the area (Western blotting, immunoenzyme assays like ELISA, protein microarray systems, etc.) including, an appropriate labelling agent.

In the present invention, the term labelling agent refers to any component that specifically determines the presence of a given component of a heterogeneous mixture or, more specifically, a biomarker. The term biomarker refers to a substance, whose, presence can be objectively measured and assessed to indicate the existence of AD either by its own presence or by modifications in its quantity or concentration.

The biomarker used in this invention can be any AD biomarker. Preferentially, βA peptide will be used, understanding βA peptide to mean the proteins or peptides that form part of the neuritic or senile plaques, both the amyloid protein and the β-amiloid protein precursor (APP), or any of its isoforms of 39 to 42 amino acid residues generated by the natural proteolytic process that gives rise to several peptides (Aβ₁₋₄₀, Aβ₂₋₄₀, Aβ₁₋₄₂) from APP.

The labelling agent selected varies according to the technique chosen to detect the presence of AD. Often, these markers are known in the state of the technique are fluorophores, that is compounds that emit florescence once joined to the biomarker so that the presence of this biomarker can be easily determined in the sample tested, For example, a fluorophore that is widely used to detect beta-amyloid is Congo red and its derivatives, which gives rise to amyloid deposits stained a shade between pink and orange that is easily detectable.

Besides using fluorophores, plaques can be identified using other compounds such as thioflavin, crystal violet, or methenamine silvers Specific anti-biomarker antibodies can also be used as labelling agents.

The kit and method of the invention represent a benefit for the patient, since its use allows the detection of an AD biomarker in vitro and not directly on the eye itself. This means that no gel or ointment needs to be applied along with the βA labelling agent to the patient's eye nor do any measurements on the eye have to be made, as occurs with some of the latest known inventions to detect βA (EP1420830A1), thus avoiding any secondary effects such as anaphylactic reactions. Moreover, in the elaboration of the method and in the detection kit, use is made of the remains of the crystalline lens produced during surgery that are normally discarded to detect the presence of βA or another biomarker of AD. Cataract operations, in which crystalline lens remains and other eye fluids are generated, are conducted in persons of similar age or even younger than the age at which the first clinical symptoms of AD appear. The present invention thus provides a tool for the early detection of this disease allowing the timely start of treatments that will prevent or delay disease progression.

### MODES OF CARRYING OUT THE INVENTION

The examples below are provided to help better understand the invention although the invention is not restricted to these examples.

### EXAMPLE 1

### Obtaining Samples

Crystalline lens remains were obtained from 42 patients aged 68 to 88 years undergoing cataract surgery by phacoemulsification. The remains were collected in the waste plastic bags normally used in this type of surgery. Each bag contained the crystalline lens and ocular fluid remains of one operation along with the physiological saline used during surgery. Samples were kept at 4° C. until the time of processing.

### EXAMPLE 2

### Separating the Solid Crystalline Lens Remains

The contents of each bag were placed in a precipitation flask for 24 h, after which as much supernatant as possible was withdrawn and the resulting sediment was centrifuged. From this first centrifugation step, 15 ml of supernatant (designated M1) were placed in a test tube and centrifuged for 15 rain at 5000 rpm. This operation rendered a pellet of solid remains and a dissolution designated M2.

From this new dissolution M2, a 5 ml volume was obtained and centrifuged in an Eppendorf tube for 10 min at 5000 rpm. This operation rendered a pellet of solid remains and a dissolution designated M3.

From this new dissolution M3, a 5 mi volume was obtained and centrifuged in an Eppendorf tube for 5 min at 5000 rpm. This operation rendered a pellet of solid remains and a dissolution designated M4.

Finally, from M4 a 5 ml volume was obtained and centrifuged in an Eppendorf tube for 5 min at 5000 rpm, This operation rendered a pellet of solid remains and a supernatant, which was discarded.

After joining together the solid remains obtained in the prior centrifugation steps, a final centrifugation was performed to eliminate all the supernatant possible rendering a compact pellet.

### EXAMPLE 3

### Paraffin Embedding of the Pellet of Crystalline Lens Fragments Separated from the Physiological Saline Dissolution

Once the pellet of solid remains was obtained, it was placed in an open-ended tube, which was covered at each end with a fine to medium fabric. The tube was then placed in a container with tap water with the tap open to constantly add water for 2 to 4 h. Once thoroughly washed, the pellet was dehydrated in alcohol.

The pellet was removed from its tube, dried with a sterile gauze and immersed in 30% alcohol for 30 min. Once this time had elapsed, the sample was again dried with a sterile gauze and immersed in 50% alcohol for 25 min. After these 25 min, the sample was dried and immersed in 70% alcohol for 24 h.

The sample was dried with a gauze and immersed in 96% alcohol in two 2 min steps. After drying the sample, it was immersed in 100% alcohol in three steps, two 25 min steps and a final step of 1 h and 30 min. The sample was dried and immersed in xylene for 40 min.

The sample was immersed in a 1:1 dissolution of liquid paraffin and xylene, for 30 min in an oven at 50° C. Following this the sample was embedded in liquid paraffin in three steps, two 45 min steps and a final step of 1 h and 30 min, During this stage the vessel with paraffin was kept in an over at 50° C.

Once the dehydration process was over; the paraffin blocks were mounted. The materials used for this process were liquid paraffin at a temperature of 50° C., Leuckart embedding irons and a Bunsen burner. Paraffin was poured into the Leuckart irons placed on a metal surface, the sample positioned and embedded in the still liquid block. The block was then introduced in a crystallizer with running water until it solidified with the sample inside. Using a microtome, 10 μm-thick sections of the paraffin block were cut. Once a series of cuts were obtained they were placed on water at 40° C. to stretch the cuts. These were then lifted onto a glass slide and placed overnight in, an incubator at 37° C. to fix them.

### EXAMPLE 4

### Staining with Congo Red in Meyer's Haematoxylin

Once the sections were prepared, they were rehydrated so that they could be stained by reversing the dehydration process described in example 3. that is, using a graded series of decreasing alcohol concentrations until the sections were immersed in deionized water.

The sections were then placed in Meyer's haematoxylin for 10 minutes, rinsed for 5 min in tap water and left in sodium chloride solution for 20 min. Next, a solution of alkaline Congo red (SIGMA-ALDRICH) was used to stain the sections for 20 min following the manufacturer's instructions.

The appearance of colour in the Congo red stained samples examined with a double polarized light microscope was used as the criterion indicating the presence of βA peptide in the sample.

### EXAMPLE 5

### Staining with Congo Red in Gill's Haematoxylin

This was conducted as in Example 4 except using Gill's haematoxylin ill for 3 min instead of Meyer's haematoxylin.

### EXAMPLE 6

### Staining with Thioflavin T

The sample section was rehydrated by reversing the dehydration process described in example 3, i.e., using a graded series of decreasing alcohol concentrations until the sections were immersed in deionized water. The deparaffinated and rehydrated sections were incubated with anti-peptide antibody in a humid chamber for 2 h at room temperature. Next, the sections were incubated with a mixture of rabbit anti IgG conjugated to Alexa-594 diluted 1:500 and thioflavin T 10 μM, both prepared in bovine serum albumin (BSA) 1% (w/v) in PBS 1X buffer, pH 7.4 for 4 h in the dark. The sections were then washed for 5 min 3 times in PBS 1X1 Tween 20 0.05% (w/v).

The sections were mounted in a medium for fluorescence microscopy and observed in an epifluorescence microscope using filters for rhodamin (594 rim) and FITC (488 nm) to visualize the red fluorescence from Alexa-594 and green fluorescence from thioflavin T.

### EXAMPLE 7

### Mounting

After staining, the sections were dehydrated and rinsed for final mounting. Dehydration was performed in an increasing series of alcohol. Rinsing was conducted in xylol. The aim of this step is to impregnate the cut in Canada balsalm solvent, which confers the sample a similar refraction index to that of glass.

For mounting, the glass slid: around the cut was cleaned and a drop of Canada balsalm dissolved in xylol applied. The slide was then covered with a coverslip. After leaving to dry for a few hours, it was observed under the microscope

### EXAMPLE 8

### Detecting βA Peptide in Paraffin Sections using Anti-Human βA Monoclonal Antibodies

The monoclonal mouse anti-human βA antibody (Dako) diluted 1:50 was applied to paraffin-embedded, formalin-axed sections. The heat-induced epitope retrieval time was 10 min and time of incubation with the primary antibody at room temperature was 30 min.

For epitope retrieval, the tissue sections were deparaffinated and rehydrated as described in example 4 and then immersed in preheated Dako Target Retrieval Solution, high pH, concentration 10× diluted 1:10 with deionized water in a water bath at 95° C. After 30 min, the container with the slides was removed from the water bath.

Once the samples had been left to cool for 20 minutes at room temperature, the high pH target retrieval solution was decanted off and the sections rinsed three times in buffer, pH 8.0, containing 5.0 M guanidineHCl and 50 mM trisHCl at ambient temperature.

As negative controls, parallel incubations were run using DakoCytomation Mouse IgG1 diluted at the same concentration as the primary antibody.

### EXAMPLE 9

### Detecting βA Petide by Immunoenzyme Assay

Levels of βA peptide were quantified by sandwich ELISA using the kit innotest A β1-42 (Innogenetics, Belgium), which detects fragment β1-42 of the amyloid protein.

The samples obtained in example 2 were solubilized in ice-cold buffer containing 5.0 M guanidineHCl and 50 mM trisHCl, pH 8.0, incubated for 3 to 4 hours and then diluted 1:10 in ice-cold buffer with casein 0.25%, sodium azide 0.05%, 20 μg/ml aprotinin, 5 mM EDTA, pH 8.0, and 10 μg/ml leupeptin in PBS. This was followed by centrifugation at 16,000 rpm for 20 min at 4° C.

Wells impregnated with the primary antibody (anti-βA) were incubated for 1 h at 37° C. with another biotinylated anti-βA antibody, the corresponding samples, peroxidase-conjugated streptavidin and a chromogen solution (tetramethylbenzidine dissolved in dimethyl sulphoxide). The reaction was stopped with 1N sulphuric acid and the absorbances of each well read at a wavelength of 450 nm.

A standard curve from 100 to 2000 pg/ml was prepared to obtain the equation and transform the absorbance data into protein concentrations (pg/ml). For each sample, the mean of duplicate determinations made in each sample was taken as the final result. Values obtained for two wells showing a difference greater than 20% were discarded.

1. A kit for the detection of Alzheimer's disease comprising:
a) a recipient in which ocular remains produced during crystalline lens surgery are collected;
b) a labelling agent that binds to a biomarker of Alzheimer's disease present in the said ocular remains collected in the said recipient.

2. A kit for the detection of Alzheimer's disease in accordance with claim 1, that also comprises a system for detecting the said labelling agent bound to the said biomarker.

3. A kit for the detection of Alzheimer's disease in accordance with claim 1, that also comprises the media required to separate the solids from the liquids contained in the recipient.

4. A kit for the detection of Alzheimer's disease in accordance with claim 1, wherein said biomarker is β-amyloid peptide.

5. A kit for the detection of Alzheimer's disease in accordance with claim 1, wherein said labelling agent is a fluorescent compound.

6. A kit for the detection of Alzheimer's disease in accordance with claim 5, wherein said fluorescent compound is Congo red or a derivative thereof.

7. A kit for the detection of Alzheimer's disease in accordance with claim 5, wherein said fluorescent compound is thioflavin T.

8. A kit for the detection of Alzheimer's disease in accordance with claim 1, wherein said labeling agent is an antibody.

9. A kit for the detection of Alzheimer's disease in accordance with claim 8, wherein said antibody is an anti- β-amyloid monoclonal antibody.

10. A kit for the detection of Alzheimer's disease in accordance with claim 1, wherein the detection system is based on animmunological technique.

11. A kit for the detection of Alzheimer's disease in accordance with claim 10, wherein said immunological technique may be sandwich ELISA, competitive ELISA, direct ELISA, indirect ELISA or Western blotting.

12. An in vitro method for the detection of Alzheimer's disease comprising:
a) a collection step for collecting the remains of crystalline lens surgery;
b) a detection step for detecting a biomarker of Alzheimer's disease in said recovered remains.

13. An in vitro method in accordance with claim 12, that comprises an additional step c) between steps a) and b), wherein said additional step c) involves labelling the biomarker of Alzheimer's disease with a labelling agent.

14. An in vitro method in accordance with claim 12 that comprises an additional step d) between step a) and step b) or between a) and step c), wherein this additional step d) involves separating the solids from the liquids in the recovered ocular remains.

15. An in vitro method in accordance with claim 12, wherein the biomarker is 13-amyloid peptide.

16. An in vitro method in accordance with claim 13, wherein the labelling of step c) is conducted using a fluorescent compound.

17. An in vitro method in accordance with claim 16, wherein said fluorescent compound is Congo red or a derivative thereof.

18. An in vitro method in accordance with claim 16, wherein said fluorescent compound is thioflavin T.

19. An in vitro method in accordance with claim 13, wherein the labelling of step c) is conducted using an antibody.

20. An in vitro method in accordance with claim 19, wherein said antibody is an anti- β-amyloid monoclonal antibody.

21. An in vitro method in accordance with claim 12, wherein the detection system is based on an immunological technique.

22. An in vitro method in accordance with claim 21, wherein said immunological technique may be sandwich ELISA, competitive ELISA, direct ELISA, indirect ELISA or Western blotting.

23. The use of the discarded remains of crystalline lens surgery to prepare an in vitro diagnostic method for Alzheimer's disease.

24. Use in accordance with claim 23, wherein said in vitro diagnostic method comprises:
a) a recovery step whereby the remains of crystalline lens surgery are recovered;
b) a detection step whereby the presence or absence of a biomarker of Alzheimer's disease is detected in said recovered remains.

25. Use in accordance with claim 24, wherein said in vitro diagnostic method comprises an additional step c) between steps a) and b) and wherein said additional step c) involves labelling the biomarker of Alzheimer's disease with a labelling agent.

26. Use in accordance with claim 24, wherein said in vitro diagnostic method comprises an additional step d) between step a) and step b) or between step a) and step c) and wherein said additional step d) involves separating the solids from the liquids in the recovered remains.

27. Use in accordance with claim 24, wherein the biomarker is β-amyloid peptide.

28. Use in accordance with claim 23, wherein the labelling of step c) is conducted using a fluorescent compound.

29. Use in accordance with claim 28, wherein said fluorescent compound is Congo red or a derivative thereof.

30. Use in accordance with claim 28, wherein said fluorescent compound is thioflavin T.

31. Use in accordance with claim 23, wherein the labelling of step c) is conducting using an antibody.

32. Use in accordance with claim 31, wherein said antibody is an anti- β-amyloid monoclonal antibody.

33. Use in accordance with claim 24, wherein the detection systems is based on an immunological technique.

34. Use in accordance with claim 33, wherein said immunological technique may be sandwich ELISA, competitive ELISA, direct ELISA, indirect ELISA or Western blotting.
